(19) United States
(12) Patent Application Publication
(10) Pub. No.: US 2024/0148995 A1
(43) Pub. Date: May 9, 2024

(54) Laryngeal Mask Airway

(71) Applicant: Medeco Pty. Ltd., Broadbeach Waters (AU)

(72) Inventors: Alexandra Anderson, Broadbeach Waters (AU); Daniel Anderson, Broadbeach Waters (AU)

(21) Appl. No.: 18/281,576

(22) PCT Filed: Mar. 11, 2022
(86) PCT No.: PCT/AU2022/050209

§ 371 Date: Sep. 12, 2023

(30) Foreign Application Priority Data
Mar. 12, 2021 (AU) 2021900718

(51) Int. Cl. A61M 16/04; A61M 16/08; A61M 39/10

(52) U.S. Cl. CPC A61M 16/0415; A61M 16/0463; A61M 16/0479; A61M 16/0875; A61M 39/10; A61M 2205/0216

(57) ABSTRACT

A laryngeal mask airway device (LMA) is disclosed comprising a ventilation conduit having operatively upper and lower ends, and a mask at the operatively lower end of the ventilation conduit. The mask has an inner surface forming a receptacle that is in fluid communication with the ventilation conduit, and an outer oropharyngeal surface facing away from the receptacle. The device includes a separate oropharyngeal suction conduit having an operatively lower end forming a collector defining a plurality of suction openings that extends across the oropharyngeal surface of the mask. In use secretions may be drawn through the suction openings into the oropharyngeal suction conduit for removal from the patient through the oropharyngeal suction conduit.

### CROSS-REFERENCE TO RELATED APPLICATIONS

This application is the national stage application of International Application No. PCT/AU2022/050209 filed Mar. 11, 2022 which designates the United States of America. This application also claims priority, under 35 U.S.C. § 119, to Australian Patent Application No. 2021900718 filed Mar. 12, 2021. The prior applications are herein incorporated by reference in their entirety.

### FIELD

This invention relates to a laryngeal mask airway device (LMA). It also extends to a method of treating a patient using a LMA device.

### Definition

In this specification, the term ‘comprising’ is intended to denote the inclusion of a stated integer or integers, but not necessarily the exclusion of any other integer, depending on the context in which that term is used. This applies also to variants of that term such as ‘comprise’ or ‘comprises’.

In this specification, the terms ‘secretion’ and ‘gastric matter’ shall be broadly interpreted. The term ‘gastric matter’ shall be interpreted to include gastric contents, gastric reflux, and gastric secretions.

Further in this specification, the term ‘cuff’ shall be broadly interpreted and shall not be limited to an inflatable cuff. In particular, the term ‘cuff’ shall be interpreted to cover both inflatable cuffs and cuffs that are not inflated.

### BACKGROUND

A laryngeal mask airway device (LMA) is a medical device that keeps a patient's airway open during anaesthesia or while they are unconscious. Another term for a LMA device is a supraglottic airway device and this term can be used interchangeably with LMA devices both in the art and in this specification.

The LMA was invented by a British anesthetist, Archibald Brain, and was commercialized in 1987. Since then, it has been widely used around the world in anaesthetics practice. The LMA device provides a treatment solution somewhere between a face mask and a tracheal intubation (where tracheal tubes are passed into a patient's trachea). An advantage of a LMA device is that it directs an artificial air supply directly to the respiratory tree with an end-to-end connection. It provides an effective artificial air supply which is delivered directly into the respiratory tract without the trauma of a full intubation.

A basic prior art LMA device is illustrated in FIG. 1. It comprises an air tube or ventilation conduit having an elliptical mask forming a receptacle at its operatively lower end, which once deployed, forms an airtight seal on top of a patient's glottis. The tube has an operatively upper end terminating in an airway connector which projects out the patient's mouth and is connected to an external air supply. As shown in FIG. 1, the air tube or ventilation conduit opens into the receptacle formed by the mask which places the receptacle in communication with the air tube. When a LMA device is correctly placed in position on a patient, a lower end or tip of the mask sits in an upper portion of the oesophagus. FIG. 2A shows a LMA device mounted in position within the throat and pharynx of a patient.

The elliptical mask has a cuff which is configured to seal against the patient's anatomy and form an airtight seal. This way, air is directed from the air tube or ventilation conduit into the patient's respiratory tract and does not leak out from the peripheral edge of the mask.

In one form, the cuff is in the form of an inflatable cuff which is inflated to seal against the patient's glottis and form an airtight connection. An inflatable cuff has an inflation line extending away from the cuff alongside the airway line that is passed out through the mouth of a patient. The inflation line has a valve outside the patient's mouth for use by a clinician to inflate the cuff. The inflation line may also have an inflation indicator for indicating to a clinician when the cuff is suitably inflated recognizing that the clinician cannot directly view the cuff received internally within the patient's body. In another form, the cuff may be made from a conformable material, e.g., a soft cushioning material, that is configured to seal against the patient's tissues adjacent the glottis.

Another example of a LMA device known in the prior art has an orogastric port (or gastric 25 drain) which opens into the oesophagus of the patient when the LMA is mounted in position on the patient. The orogastric port has an open lower end facing down into the oesophagus and an upper end which is positioned outside the mouth of the patient. The orogastric port runs parallel to the ventilation conduit and does not open into the ventilation conduit.

The orogastric port provides broadly a basic conduit in parallel with the ventilation conduit for inserting a catheter therethrough to facilitate the removal or gastric contents or secretions from the patient.

The catheter is inserted through the orogastric port which functions as a guide conduit and down through the operatively lower end of the orogastric port through the oesophagus towards the stomach of the patient. A suction may be applied to the catheter to draw secretions up through the orogastric port and remove them from the body.

The catheter in the orogastric port facilitates removal of gastric secretions and/or gastric reflux from the oesophagus and stomach of the patient while the LMA device is in use with the mask mounted over the glottis. It draws this material from a position spaced beneath the mask of the LMA device.

Another LMA device known in the prior art has a bite block and a tab towards its operatively upper end adjacent to the airway connector. This enables the patient to bite on the bite block when the LMA is mounted in position on the patient.

However, even though the use of a LMA has been greatly improved since it first appeared, it still has shortcomings. For example, there is a risk of aspiration of secretions, such as blood from operative field/s in the naso or oropharyngeal region or saliva, into the lungs when an LMA is removed. Secretions pool on the superior (oropharyngeal aspect or surface) of the LMA and this can end up on the glottis when the LMA is removed. Secretions that fall on the glottis, particularly on emergence (i.e., during a lighter plane of anaesthesia) can trigger airway crises such as laryngospasm and/or bronchospasm.

These shortcomings lead to increased risk and, in some instances where the risk is assessed as unacceptably high, the clinician may decide to use endotracheal intubation instead. However, intubation is more traumatic than the use of a LMA device and typically involves the use of a muscle relaxant which increases the risk of anaphylaxis, inadequate reversal, and awareness under anaesthesia. A muscle relaxant would often be used in a person weighing more than 30 kg.

The reference to prior art in the background above is not and should not be taken as an acknowledgment or any form of suggestion that the referenced prior art forms part of the common general knowledge in Australia or in any other country.

### SUMMARY OF THE INVENTION

Applicant recognizes that it would be beneficial if a laryngeal airway mask device could be devised that ameliorated at least some of the drawbacks identified above for prior art LMA devices. It would be particularly advantageous if it could facilitate the removal of secretions pooling on an oropharyngeal surface of a mask to reduce the risk of an airway crisis occurring with the patient. An airway crisis can be life threatening and can lead to a death or an ICU admission. Additionally, Applicant believes that it would be beneficial if the LMA device could be made easier for clinicians to use as this would enable them to respond more quickly in an emergency.

According to one aspect of this invention there is provided a laryngeal mask airway device (LMA), comprising: a ventilation conduit having an operatively upper end and an operatively lower end; a mask at the operatively lower end of the ventilation conduit, the mask having an inner surface forming a receptacle that is in fluid communication with the ventilation conduit and an outer oropharyngeal surface (or posterior surface) opposed to said inner surface and facing away from the receptacle; and a separate oropharyngeal suction conduit having an operatively upper end, and an operatively lower end forming a collector defining a plurality of suction openings that extends across the oropharyngeal surface of the mask, wherein in use secretions are drawn through the suction openings into the oropharyngeal suction conduit for removal from the patient through the oropharyngeal suction conduit.

By using suction to remove these secretions, aspiration of the secretions into the lower airways, and the complications caused by aspiration, can be resisted. Some potential complications of the lower airways include laryngospasm and bronchospasm, and these conditions are particularly prevalent when a patient emerges from anaesthesia.

The collector may extend across substantially the full oropharyngeal surface of the mask, and the plurality of suction openings may be spaced apart from each other across the oropharyngeal surface. That is, the collector may extend across the full extent of the oropharyngeal surface to gather secretions from across the full oropharyngeal surface of the mask.

The mask may be configured in the form of an ellipse and may be referred to as an elliptical mask.

The LMA device may include a cuff extending around a peripheral edge of the mask for sealing the mask to a patient, e.g., the glottis of a patient.

The collector may have a perimeter region extending along at least part of the perimeter of the collector, and a raised inner region inward of the perimeter region. The raised inner region may slope down to the perimeter region on each side of the raised line.

The collector may include a first plurality of suction openings spaced apart from each other along the perimeter region. Conveniently, the first plurality of suction openings may be arranged in a line along the perimeter region.

The collector may also include a further plurality of suction openings on the raised inner region, e.g., on either side of the raised line extending substantially the length of the collector.

The suction openings may be arranged and positioned relative to each other on the collector such as to provide the collector with sufficient structural integrity.

The collector may further include a recess extending along a part of the length of the perimeter region for pooling and/or directing secretions towards the suction openings in the perimeter region.

In particular, the recess may form an open channel extending along substantially the full length of the perimeter region. In particular, the open channel may be formed with curved surfaces and rounded edges (with no square or obtrusive edges).

The oropharyngeal suction conduit may include a suction control for enabling a clinician to selectively apply a suction to the oropharyngeal suction conduit.

The operatively upper end of the oropharyngeal suction conduit may include a pipe coupling formation for releasable coupling to an external suction conduit.

In one form, the suction control may be in the form of a branch pipe on the pipe coupling formation having an open end that is normally open to the outside air for drawing external air into the oropharyngeal suction conduit.

The open end of the branch pipe may be closed, e.g., by a clinician applying a finger over the open end to apply a suction to the oropharyngeal suction conduit and draw fluid and secretions in through the collector at the operatively lower end of the oropharyngeal suction conduit.

The pipe coupling formation may comprise a male pipe coupling formation for releasable connection to a complementary female pipe coupling formation on the external suction conduit.

The male pipe coupling formation may be in the form of a spigot having one or more transverse ribs or gripping formations and the female pipe coupling formation on the external suction conduit may be in the form of a complementary socket. The rib or gripping formations may be configured to releasably engage the female pipe coupling formation.

In use, this facilitates detachment of the pipe coupling formation from, and reattachment of the pipe coupling formation to, the external suction conduit. This enables the coupling formations to be manually connected to each other by a clinician during operation of the LMA device.

The external suction conduit may be operatively coupled to a suction pump.

The laryngeal mask may further include an orogastric conduit having an operatively upper end and an operatively lower end, for removing gastric matter from the patient.

The orogastric conduit or orogastric port (or gastric tube) facilitates removal of gastric matter from the stomach or oesophagus of the patient during use of the LMA device.

The orogastric conduit is separate from the oropharyngeal suction conduit and does not open into or communicate with the oropharyngeal suction conduit along its length. In particular, the orogastric conduit has no openings intermediate the upper and lower ends that would permit gastric matter or secretions to be discharged from the orogastric conduit in proximity to the collector on the mask. This keeps any gastric contents that are drawn up through the gastric conduit quite separate from secretions drawn up through the oropharyngeal suction conduit.

The orogastric conduit may include an orogastric pipe coupling formation for releasable coupling to an external suction conduit.

For example, the orogastric pipe coupling formation may comprise a male pipe coupling formation for releasably connecting to a complementary female pipe coupling formation on the external suction conduit whereby in use to facilitate detachment of the orogastric pipe coupling formation from, and reattachment of the orogastric pipe coupling formation to, the external suction conduit.

The orogastric pipe coupling formation may include a suction control for enabling a clinician to selectively apply a suction to the orogastric conduit. The suction control may be similar to that described above for the suction control on the oropharyngeal suction conduit.

The oropharyngeal suction conduit and the orogastric port may be interchangeably coupled to the external suction conduit in use, i.e., so that a single external suction line may be used to provide suction to both.

Thus, the external suction conduit may provide suction to both the oropharyngeal suction conduit and the orogastric port, it being recognized that suction is only applied intermittently by the clinician on each of the oropharyngeal suction conduit and the orogastric port.

Additionally, the orogastric conduit may be configured to form a port for receiving a catheter therethrough for withdrawing gastric matter from the patient. That is, in addition to serving as a conduit for gastric matter, the conduit may also receive a small catheter therein which is fed down through the port and into. Such a catheter may be a small catheter, e.g., an 8 Fr catheter down to a 12 Fr port.

The cuff may be an inflatable cuff that is inflated for sealing the mask to the body of a patient, and the cuff may further include an inflation line extending away from the cuff for inflating the cuff.

The cuff may further include a valve on the inflation line which can be operated to introduce air under pressure into the cuff to inflate the cuff. The inflation line may also include an inflation indicator for indicating to a clinician when the cuff is suitably inflated.

Instead, the cuff may be a conforming cuff or seal formed of a conformable cuff material that conforms to and seals against the body of a patient without being inflated.

That is, the cuff material conforms and adapts to the shape of the surface of the anatomical structures of the patient against which it abuts and seals against the body. The properties of the cuff material enable it to conform and seal up against the anatomy of the patient.

The oropharyngeal suction conduit may extend parallel to the ventilation conduit outside of the ventilation conduit. Optionally, the ventilation conduit, and the oropharyngeal suction conduit may be contained within a common housing along their lengths.

The common housing may have smooth rounded surfaces free of any protruding surfaces that would interfere with any internal part of the body of a patient. Optionally, the common housing may have an oval or circular cross-sectional shape.

In embodiments where the LMA device includes an orogastric conduit, the orogastric conduit may also extend parallel to the ventilation conduit outside of the ventilation conduit. Further, the orogastric conduit may also be contained within the common housing containing the ventilation conduit and the oropharyngeal suction conduit.

The ventilation conduit and the oropharyngeal suction conduit and optionally also the orogastric conduit may each be formed by a tube.

Further, the ventilation conduit, the oropharyngeal suction conduit and the orogastric conduit may each have some flexibility or bendability.

The ventilation conduit and the oropharyngeal suction conduit, and optionally also the orogastric conduit, may be formed from a suitable medical grade material, e.g., PVC, silicone rubber, a thermoplastic polymer, and/or a bioplastic.

The LMA device may further include a bite block adjacent an upper end of the ventilation conduit providing a rigid member for the patient to bite on when the LMA device is inserted into a patient.

The laryngeal mask airway device may include any one or more of the features, or combination of features, of the laryngeal mask airway device in any other aspect of the invention in the summary section.

According to another aspect of the invention there is provided a laryngeal mask airway device (LMA), comprising: a ventilation conduit having an operatively upper end and an operatively lower end; a mask at the operatively lower end of the ventilation conduit, the mask having an inner surface forming a receptacle that is in fluid communication with the ventilation conduit and an outer oropharyngeal surface opposed to said inner surface and facing away from the receptacle; and a separate oropharyngeal suction conduit having an operatively upper end and an operatively lower end forming a collector on the oropharyngeal surface of the mask for enabling a clinician to remove secretions from the oropharyngeal surface, the operatively upper end comprising a pipe coupling formation for releasably coupling the suction conduit to an external suction conduit.

The device may include a cuff extending around a peripheral edge of the mask for sealing the mask to a patient.

The pipe coupling formation may comprise a male pipe coupling formation for releasable connection to a female pipe coupling formation on the external suction conduit.

The oropharyngeal suction conduit may include a suction control towards its operatively upper end for enabling a clinician to selectively apply a suction to the suction conduit.

The suction control may be in the form of a branch pipe on the male pipe coupling formation having an open end that is normally open to the outside air for drawing external air into the oropharyngeal suction conduit, wherein the open end of the branch pipe may be closed to apply a suction to the oropharyngeal suction conduit and draw secretions into the collector.

The open end of the branch pipe may be closed in use by a clinician applying a finger over the open end to apply a suction to the oropharyngeal suction conduit and draw secretions through the collector into the operatively lower end of the oropharyngeal suction conduit.

The laryngeal mask airway may include any one or more of the features, or combination of features, of the laryngeal mask airway in any other aspect of the invention in the summary section.

According to yet another aspect of the invention there is provided a laryngeal mask airway device (LMA), comprising: a ventilation conduit having an operatively upper end and an operatively lower end; a mask at the operatively lower end of the ventilation conduit, the mask having an inner surface forming a receptacle that is in fluid communication with the ventilation conduit and an outer oropharyngeal surface opposed to said inner surface and facing away from the receptacle; and a separate oropharyngeal suction conduit having an operatively upper end, and an operatively lower end forming a collector on the oropharyngeal surface of the mask for enabling a clinician to remove secretions from the oropharyngeal surface, the operatively upper end comprising a pipe coupling formation for releasable coupling to a complementary pipe coupling formation on an external suction conduit; and a separate orogastric conduit in parallel with the oropharyngeal suction conduit having an operatively lower end and an operatively upper end, and an orogastric pipe coupling formation on the operatively upper end for releasable coupling to a complementary pipe coupling formation on an external suction conduit, wherein the pipe coupling formations of the oropharyngeal suction and orogastric conduits are configured to engage the same complementary pipe coupling formation on an external suction conduit, whereby to enable the oropharyngeal suction and orogastric conduits to be interchangeably coupled to a single external suction conduit.

The male and female coupling formations on each of the oropharyngeal suction and orogastric conduits may be configured so that they can be manually detached or separated from each other during operation of the LMA device, and also manually connected to each other by a clinician during use of the LMA device.

This interchangeability is useful because anaesthetic and surgical suction have common connecting or coupling arrangements in an operating theatre, and it is only the delivery devices that are different. This thereby enables a clinician to selectively apply suction on each of the suction and orogastric conduits using an external conduit that is already present.

The LMA device may further include an external suction conduit that is operatively connectable to the pipe coupling formations of each of the oropharyngeal suction and orogastric conduits, and the external or further suction conduit may in turn be operatively coupled to a suction pump.

The pipe coupling formations of the oropharyngeal suction and orogastric conduits may be male pipe coupling formations that are configured to be received within a standard female pipe coupling formation on the external suction conduit.

The oropharyngeal suction and the orogastric conduits may each have a suction control towards their operatively upper ends for enabling a clinician to selectively apply a suction to the suction conduit.

In one form, the suction control may be in the form of a branch pipe on the male pipe coupling formation having an open end that is normally open to the outside air for drawing external air into the suction conduit. When open, it cancels the suction by equilibrating with atmospheric pressure.

The open end of the branch pipe on each suction control may be closed, e.g., by a clinician applying a finger over the open end to apply a suction to either of the oropharyngeal suction or orogastric conduits and draw fluid and secretions in through the respective conduit.

The laryngeal mask airway device may include a cuff or seal extending around a peripheral edge of the mask for sealing the mask to a patient.

The laryngeal mask airway device may include, any one or more features, or combination of features, of the laryngeal mask airway described in any other aspect of the invention in the summary section.

According to another aspect of this invention there is provided a mask for a laryngeal mask airway device (LMA), the mask comprising an air passage wall that is positioned adjacent to the glottis of a patient in use; an oropharyngeal wall extending across the air passage wall spaced away from the air passage wall and defining an oropharyngeal space therebetween, the oropharyngeal wall defining a plurality of suction openings and forming a collector for receiving secretions through the suction openings into the oropharyngeal space; and at least one internal support for supporting the oropharyngeal wall in fixed spaced relation relative to the air passage wall.

The plurality of suction openings may be distributed across the surface of the oropharyngeal wall for collecting secretions from across the full surface thereof.

Each of the air passage wall and the oropharyngeal wall may have a perimeter and the internal support/s may be spaced inward from the perimeter of each of these walls.

The at least one internal support may comprise a single internal support, e.g., substantially centrally within the oropharyngeal space.

Instead, the at least one internal support may comprise a plurality of internal supports.

The mask may further include a side wall extending between the air passage wall and the oropharyngeal wall around the perimeter of the walls.

The air passage wall, the oropharyngeal wall, and the at least one internal support, may be integrally formed, e.g., by a moulding operation such as an injection moulding operation.

Optionally, the side wall may also be integrally formed with the air passage wall and the oropharyngeal wall.

The mask may further include a cuff extending around the perimeter of the air passage wall and the oropharyngeal wall.

The cuff may be a conforming cuff or seal and the conforming cuff or seal may be integrally formed with the air passage wall and the oropharyngeal wall.

In this form, the conforming cuff may be integrally formed with the air passage wall and the oropharyngeal wall, e.g., by a moulding operation such as an injection moulding operation.

Instead, the cuff may be an inflatable cuff mounted on the air passage wall and the oropharyngeal wall which are formed separately to the cuff.

In this form, the inflatable cuff may be mounted on the air passage wall and the oropharyngeal wall, and optionally also the side wall.

The mask may include any one or more of the other features of the masks described in the LMA devices described in any preceding aspect of the invention.

According to another aspect of this invention there is provided a mask for a laryngeal mask airway device (LMA), the mask comprising: an air passage wall that is positioned adjacent to the glottis of a patient in use; an oropharyngeal wall extending across the air passage wall spaced away from the air passage wall and defining an oropharyngeal space therebetween, the oropharyngeal wall defining a plurality of suction openings and forming a collector for drawing secretions through the suction openings into the oropharyngeal space; at least one internal support for supporting the oropharyngeal wall in fixed spaced relation relative to the air passage wall; and a conforming cuff or seal extending around a perimeter of the air passage wall and the oropharyngeal wall, wherein the air passage wall, the oropharyngeal wall, the at least one internal support, and the conforming cuff or seal are integrally formed.

The invention also extends to an LMA device comprising: a ventilation conduit having an operatively upper end and an operatively lower end; a mask as defined in any preceding aspect of the invention; and an oropharyngeal suction conduit having an operatively upper end, and an operatively lower end leading to the oropharyngeal wall, wherein in use secretions are drawn through the suction openings into the oropharyngeal space and then sucked up the oropharyngeal suction conduit for removal.

The LMA device may include any one or more of the other features of the LMA device described in any preceding aspect of the invention.

According to yet another aspect of the invention there is provided a method of providing an air supply directly into a patient's respiratory tree including, fitting a LMA device as claimed in any one of the other statements of invention, and directing air through the ventilation conduit into the patient's respiratory tree.

The method may include applying suction intermittently to the oropharyngeal suction conduit to remove secretions from the oropharyngeal surface of the mask by sucking them up through the oropharyngeal suction conduit.

The clinician may apply the suction intermittently as and when required by their operation of a manual suction control on the oropharyngeal suction conduit.

Directing air into the ventilation conduit may comprise introducing air under positive pressure into the operatively upper end of the ventilation conduit.

The method may include fitting the LMA device to a patient for carrying out oral surgery or upper airway surgery. The method may also include fitting the LMA device to a patient during emergency situations, for example, when the patient is unconscious.

The method removes secretions, e.g., secretions secreted by the patient, from the oropharyngeal surface of the LMA device so the secretions do not enter the trachea of the patient, which reduces the risk of respiratory complications including bronchospasm and laryngospasm.

The LMA device may include any one or more of the other features of the LMA device described in any preceding aspect of the invention.

For example, the LMA device may also include an orogastric conduit and the method may include feeding a catheter down through the orogastric conduit to allow intermittent suction of gastric contents through the orogastric conduit.

### BRIEF DESCRIPTION OF DRAWINGS

A LMA device and a method of using a LMA device to treat a patient in accordance with this invention may manifest itself in a variety of forms. It will be convenient to hereinafter describe several embodiments of the invention in detail with reference to the accompanying drawings. The purpose of providing this detailed description is to instruct persons having an interest in the subject matter of the invention how to carry the invention into practical effect. However, it is to be clearly understood that the specific nature of this detailed description does not supersede the generality of the preceding broad description. In the drawings:

FIG. 1 is a perspective view of a laryngeal mask airway device (LMA) known in the prior art;

FIG. 2A is a sectional view of a LMA device like that in FIG. 1 mounted in position on a patient showing the mask mounted over the glottis;

FIG. 2B is a set of schematic drawings showing the human anatomy around the entrance to the tracheal passages;

FIG. 3 is an upper perspective view of a LMA device in accordance with one embodiment of the invention;

FIG. 4 is a lower perspective view of the LMA device in FIG. 3;

FIG. 5 is a side view of the LMA device in FIG. 3;

FIG. 6 is a front view of the LMA device in FIG. 3;

FIG. 7 is a bottom plan view of the LMA device in FIG. 3;

FIGS. 8a, 8b, 8c comprises three cross-sectional views through the mask of the LMA device in FIG. 3, taken at three different positions along the length of the mask;

FIG. 9 is an upper perspective view of a LMA device in accordance with a second embodiment of the invention;

FIG. 10 is a lower perspective view of the LMA device in FIG. 9;

FIG. 11 is a side view of a part of the LMA device of FIG. 9 showing a suction conduit and collector that enables secretions from the patient to be selectively sucked up by the LMA device;

FIG. 12 is a cross sectional view of the part of the LMA device in FIG. 11; and

FIG. 13 is an upper perspective view of the part of the LMA device of FIG. 11.

### DETAILED DESCRIPTION

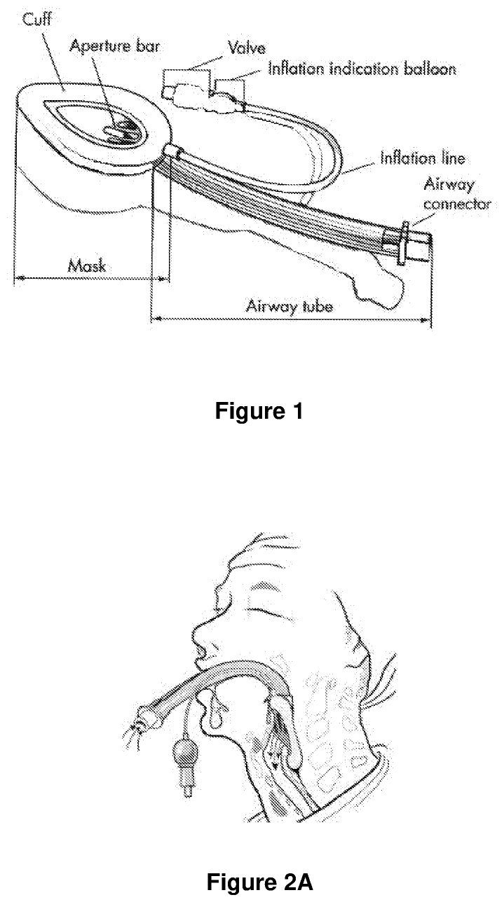

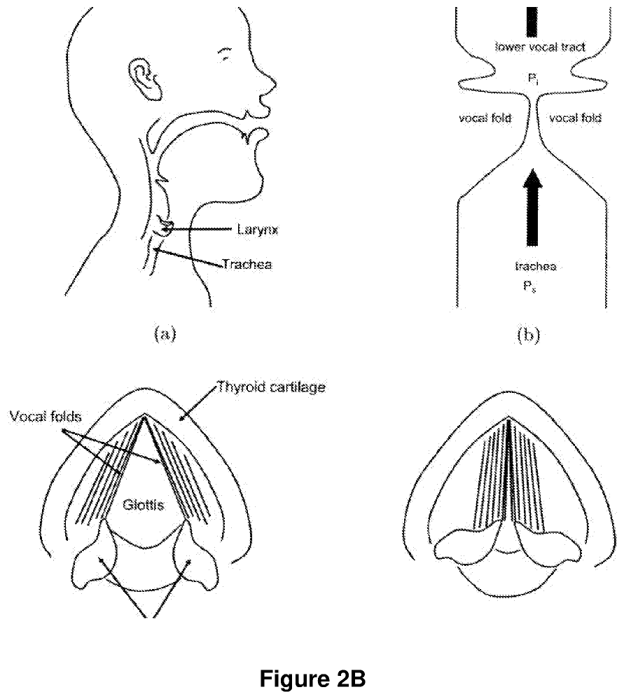

FIGS. 1 and 2 illustrate prior art LMA devices that have been discussed in the background section above. These drawings will therefore not be discussed further in the detailed description which will focus on embodiments in accordance with the invention.

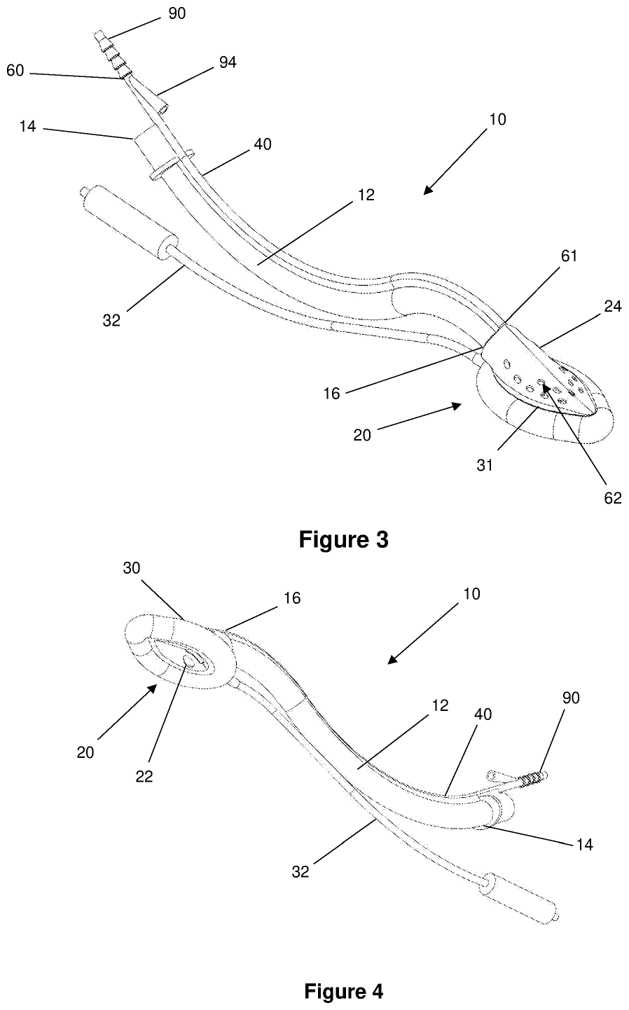

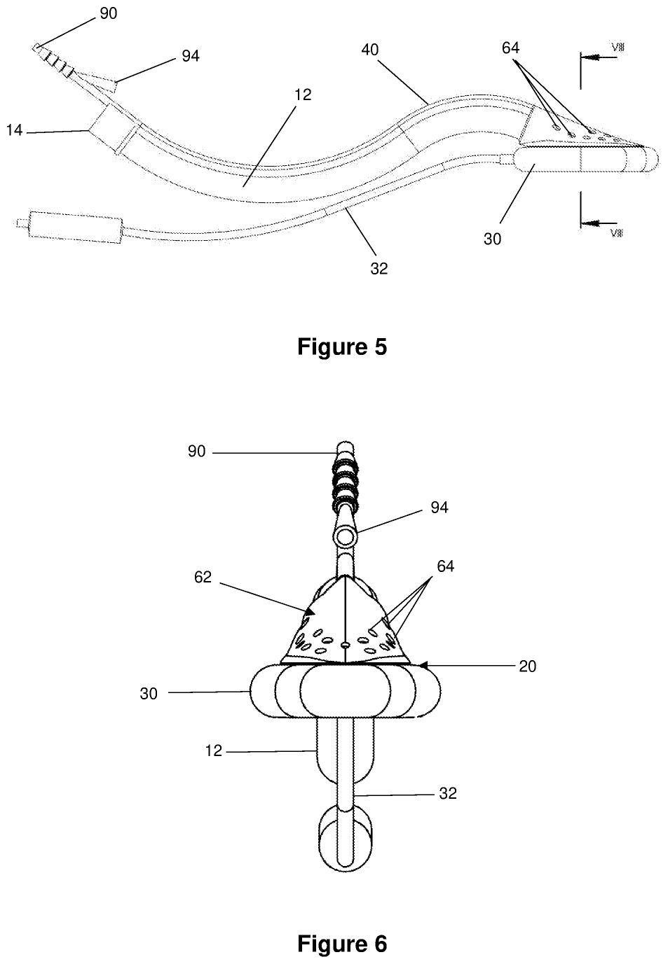

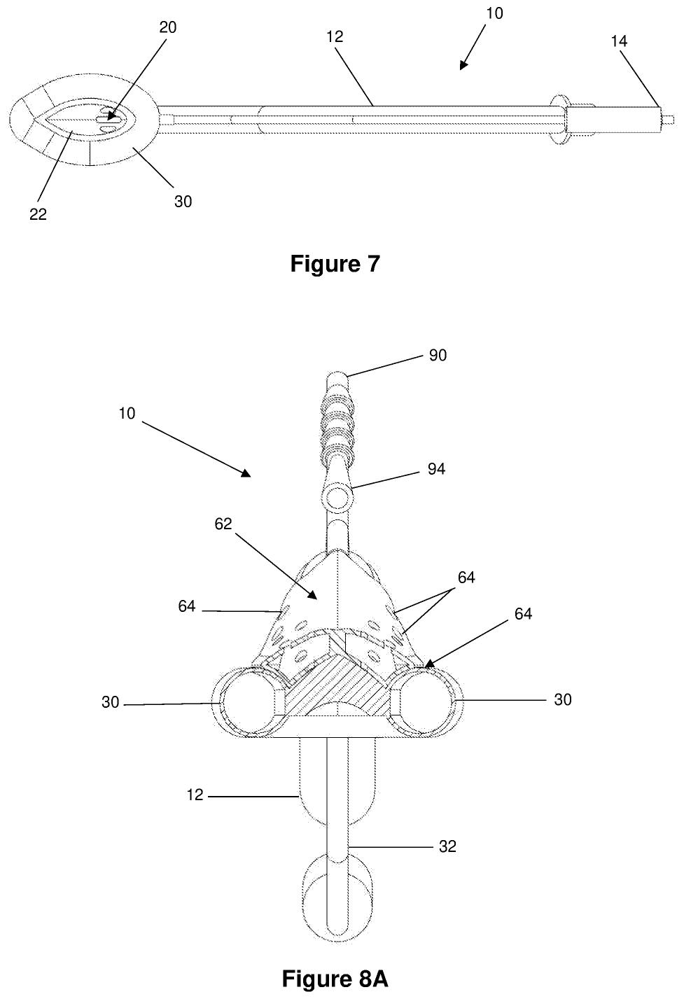

FIGS. 3 to 8 illustrate a LMA device in accordance with one embodiment of the invention. This LMA is just one example style of a LMA which has an inflatable cuff with a pressure indicator balloon for indicating an inflation pressure in the cuff.

The LMA device 10 comprises broadly an airway line or air tube or ventilation conduit 12 having an operatively upper end 14 and an operatively lower end 16, and an elliptical mask 20 at the operatively lower end 16 of the airway line 12. The elliptical mask has an inner surface 22 forming a receptacle that opens into the airway line 12 and an outer or oropharyngeal surface 24 opposed to said inner surface 22 and facing away from the receptacle. The mask 20 has a cuff 30 extending around its peripheral edge 31 for sealing the mask 20 against an operatively upper open end of the patient's airway, e.g., across the glottis of the patient.

The LMA device 10 also includes a suction conduit 40 separate from, and in parallel with, the ventilation conduit 12 that enables a clinician to apply intermittent suction for removing body secretions from the oropharyngeal surface 24 of the mask 20. The term ‘suction conduit’ 40 may be used interchangeably in this specification with the term oropharyngeal suction conduit.

Each of these components of the LMA device 10 will now be described in more detail below.

The ventilation conduit 12 is formed of a tubular material having some bendability or flexibility and is sized to be comfortably received within the mouth and throat of a patient. The operatively upper end 14 of the ventilation conduit 12 has an airway connector (like that shown in FIG. 1) that facilitates its operative connection to an external air supply apparatus. As the structure and function of an external air supply apparatus would be known to persons skilled in the art, it will not be described in further detail in this specification.

The elliptical mask 20 is sized and configured to fit over the glottis of a patient and seal the mask 20 to the patient's anatomy around the glottis so that air displaced down through the ventilation conduit 12 is directed into the respiratory tree of the patient and not into the oesophagus along a pathway leading to the stomach.

The inflatable cuff 30 extends around the peripheral edge 31 of the mask 20 for sealing against the patient's glottis and is inflated with air to urge it into sealing contact with the glottis. The inflatable cuff 30 includes an inflation line 32 extending away from the cuff 30 which typically has a valve (not shown) which can be operated to allow air under pressure into the inflation line 32 to inflate the cuff 30. The inflation line 32 typically also includes an inflation indicating balloon (not shown) for indicating to a clinician when the cuff 30 is suitably inflated.

The suction conduit 40 has an operatively upper end 60 which is positioned outside the mouth of a patient in use, and which is operatively coupled to an external suction conduit 70 (not shown in FIG. 3) and an operatively lower end 61. A suction control is operated by a clinician to create suction in the suction tube 40. The suction apparatus that is used to create the suction is known in the art and accordingly will not be described in greater detail in this description.

As shown in the drawings, the upper end 60 of the conduit 40 has a pipe coupling formation 90 thereon that can be used to releasably couple the conduit 40 to the external conduit 70. Conveniently, the pipe coupling formation 90 comprises a male pipe coupling formation that is received within a complementary female pipe coupling formation 92 on the external suction conduit 70, e.g., with a friction fit. Further, the suction control is provided by the male pipe coupling formation 90 which has a branch line 94 with an open end which can be used by the clinician to apply suction intermittently as and when required. Conveniently, the male and female pipe coupling formations 90, 92 may be similar to those used in the catheter arts.

The operatively lower end 61 of the suction tube 40 forms a collector 62 that extends (spreads out) over substantially the full oropharyngeal or posterior surface 24 of the mask 20. The collector 62 comprises a perimeter region extending around the peripheral edge 31 of the mask 20, and a raised inner region that slopes down to the perimeter region on each side of the collector 62. The raised inner region has a ridge or high line or spine extending along a length of the collector 62 and slopes down to the perimeter region on each side of the high line.

The collector 62 includes a plurality of suction openings 64 that are arranged on the oropharyngeal surface. Some of the suction openings 64 will be arranged around the peripheral edge 31, and other suction openings 64 may be arranged on the raised inner region. In the illustrated embodiment, the collector 62 has at least eight suction openings 64 arranged in a line and spaced apart from each other along the perimeter region.

Applicant points out that the number of suction openings 64 on the collector 62 in this embodiment is just one example form of an arrangement of suction openings 64. In some other forms, there may be fewer suction openings 64 on the collector 62 and they may be arranged differently thereon. In some other forms, the collector 62 may have more suction openings 64 than those indicated in FIGS. 3 to 8. For any given LMA device design, the number of suction openings 64, and the arrangement of these openings 64, will depend on the volume of secretions to be drained through the suction openings 64 in use and also the structural strength that is required from the collector 62.

One factor in the arrangement of the suction openings 64 on the collector 62 is a desire to avoid suctioning the mucosa directly onto the suction openings 64, which would occlude or block the openings 64 and also potentially cause some tissue trauma to the patient.

Additionally, the perimeter region may be formed with a recess or open channel 67 that extends around a substantial portion of the peripheral edge 31 of the mask 20. The channel 67 helps to pool secretions and direct them towards and into the suction openings 64 formed in the perimeter region. The channel 67 may be formed with curved or rounded edges only and does not have any square edges. Forming the suction openings 64 in the recess 67 may avoid suction trauma to mucosal tissue extending across the collector 62. While an open channel is not shown clearly in FIGS. 3 to 8, a person skilled in the art will recognize that the oropharyngeal surface 24 could be configured to provide a more pronounced channel.

The collector 62 includes a further plurality of suction openings 64 on the raised inner region thereof, e.g., at least one suction opening 64 on each side of the high line. In the embodiment illustrated in FIGS. 3 to 8, the further plurality of openings 64 comprises a couple of suction openings on each side of the high line towards the end remote from air tube or ventilation conduit 12, i.e., distal from the operatively upper end 14 of the ventilation conduit 12.

Thus, the collector 62 spreads out over the posterior or oropharyngeal surface 24 of the mask 20 and draws body secretions from a patient into the suction openings 64 across the full oropharyngeal surface 24. The body secretions are drawn through the suction openings 64 and then sucked up through the oropharyngeal suction conduit 40 away from the glottis at the entrance to the trachea.

The collector 62 has a low profile, as shown in the drawings, and does not occupy much space over and above the rest of the LMA device. Additionally, the collector 62 only has curved surfaces and rounded edges (with no square edges or corners), so that it does not inflict any trauma on the body tissues of the patient. This is particularly the case when the LMA device 10 is inserted into and withdrawn from the patient, in addition to when it is in position.

The oropharyngeal suction conduit 40 follows the curvature of the ventilation conduit or air tube 12 along its length and is typically made of a similar material to the ventilation conduit 12. As shown in the drawings, the suction conduit 40 has a considerably smaller diameter than the ventilation conduit 12. By way of example only, the air tube or ventilation conduit 12 may have a diameter of about 15 mm and the suction conduit 40 may have a diameter of about 5 mm. As a result, the ventilation conduit 12 and associated suction conduit 40 form a relatively streamlined object having a low profile as the conduits extend up from the glottis through the oropharyngeal passage within the patient. The LMA device 10 with its collector 62 oropharyngeal suction conduit 40 fits comfortably within the internal space formed by the oropharyngeal passage of the patient.

It will be appreciated by persons skilled in the art that the size of tubing used for both the ventilation conduit 12 and the suction conduit 40 will vary depending on the size of the LMA device 10, i.e., a larger sized LMA device 10 will generally have a larger ventilation conduit 12.

In the illustrated embodiment in FIGS. 3 to 8, the ventilation conduit 12 and the suction conduit 40 are held together along their length. However, it will readily be appreciated by a person skilled in the art that the conduits 12 and 40 also could extend from their upper ends to their lower ends independently and without being attached to each other.

The LMA device in FIGS. 3 to 8 does not have an orogastric port like that described above in the Background section on a prior art LMA device. That is, it does not have a separate orogastric port in parallel with the ventilation conduit 12 and the oropharyngeal suction conduit 40 that can be used to withdraw gastric contents and secretions from the stomach and/or oesophagus of the patient.

In use, a LMA device 10 like that shown in FIGS. 3 to 8 is mounted on a patient to provide an air supply direct to their respiratory tree. The glottis marks the entrance to the respiratory tree, and the point at which the food passageway from the mouth to the stomach diverges from the respiratory pathway. LMA devices are used by medical clinicians having a variety of backgrounds and specializations. For example, they may be used by a consultant anesthetist doctor and sometimes they may be removed by a recovery nurse.

The LMA device 10 provides a direct end to end connection from an air supply (external 20 to the patient) to an entrance of the respiratory tree. FIG. 2B shows the relevant anatomy of the patient including the pharyngeal cavity and the glottis over which the LMA device is mounted. The LMA device 10 is inserted through a patient's mouth into their throat and then displaced down to a position where the anterior or cup shaped inner surface 22 of the mask 20 (opposite the oropharyngeal surface 24) is positioned across the patient's glottis facing the patient's vocal cords. The cuff 30 which is by then positioned around the glottis is inflated to urge the cuff 30 into sealing contact against the glottis to form an airtight seal. That way, air directed down the air tube or ventilation conduit 12 is passed directly into the respiratory tree for oxygenating blood passing though the lungs and does not leak out of the mask 20.

During use of the LMA device 10, the suction conduit 40 can be used by an attending clinician to suction out upper airway secretions and blood that may collect in the body cavity or space around the oropharyngeal surface 24 of the mask 20. The clinician can apply suction on an intermittent basis as required, e.g., by manual activation of the suction control at the operatively upper end 60 of the suction conduit 40 and external to the patient. The plurality of suction openings 64 in the collector 62 draws secretions from across the full extent of the oropharyngeal surface 24 into the suction conduit 40. When suction is applied, the secretions are drawn up through the suction conduit 40 and into an external suction conduit 70 and from there they are passed into a canister/reservoir.

After airways treatment, i.e., the delivery of oxygen into the patient's lungs has been completed, the LMA device 10 can then be displaced up through the pharynx and throat and be withdrawn through the mouth. This is done manually by holding onto the airway connector or similar. With current techniques the cuff is still inflated when the LMA device is withdrawn. This helps to avoid secretions dripping down onto the larynx. Applicant believes that there may be scope to deflate the cuff for withdrawal when secretions are suctioned off the oropharyngeal surface as disclosed in this application.

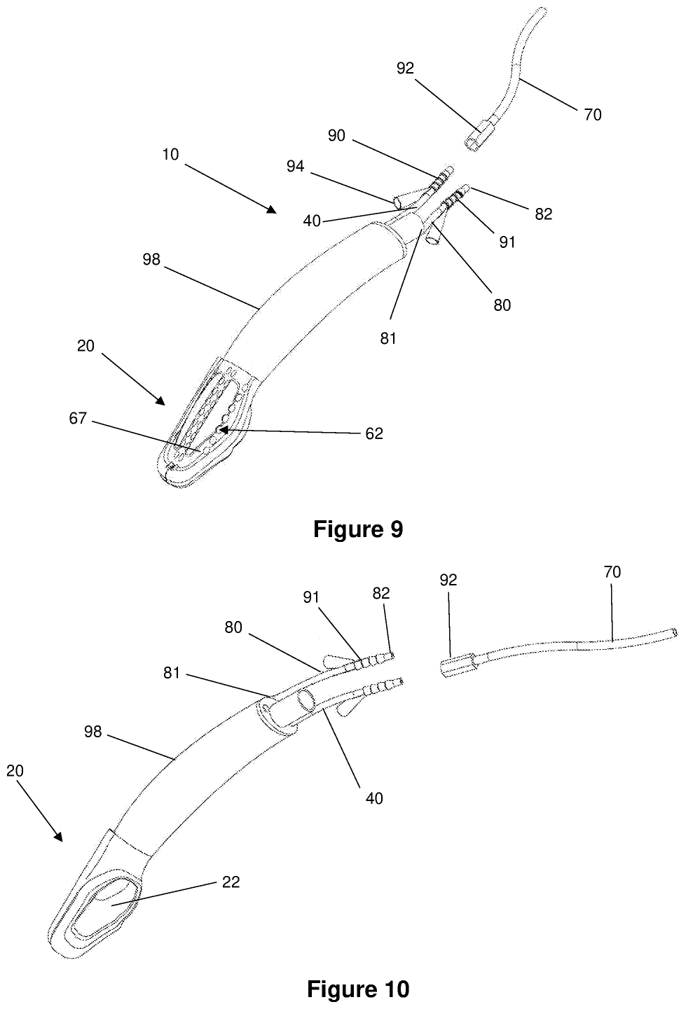

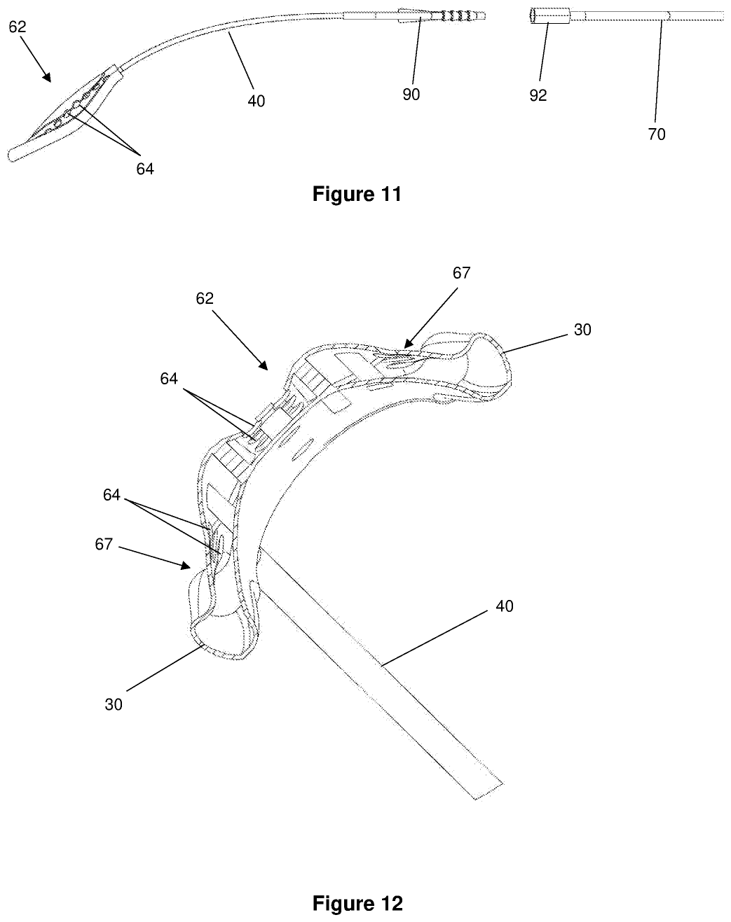

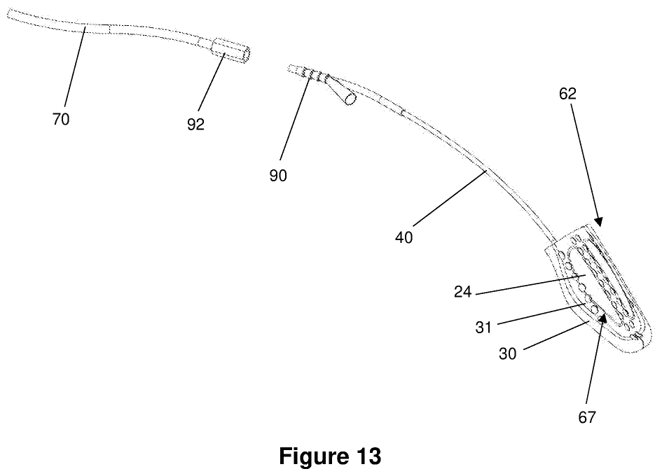

FIGS. 9 to 13 illustrate an LMA device in accordance with another embodiment. As the LMA device has some similarities to the LMA device shown in FIGS. 3 to 8 above, the same reference numerals will be used to refer to the same components unless otherwise indicated. Further, the following description will focus on the differences between this embodiment and the FIG. 3 embodiment.

The design of the LMA device 10 shown in FIGS. 9 to 13 is similar to the design of an ‘i-gel’ LMA device produced by Intersurgical Pty Ltd. In this embodiment, the basic ‘i-gel’ LMA product has been adapted to provide a suction conduit 40 extending in parallel to the air tube or ventilation conduit 12 for enabling a clinician to remove body secretions and mucus in proximity to the oropharyngeal surface 24 of the mask 20.

Broadly, the LMA device 10 comprises an airway line or air tube or ventilation conduit 12 having an operatively upper end 14 and an operatively lower end 16, and an elliptical mask 20 at the operatively lower end 16 of the ventilation conduit 12. The elliptical mask 20 has an inner surface 22 forming a receptacle that opens into the ventilation conduit 12 and an outer or oropharyngeal surface 24 opposed to said inner surface 22 and facing away from the receptacle.

In this embodiment, the mask 20 has a conforming cuff or seal 30 extending around its peripheral edge 31 for sealing the mask 20 against an operatively upper open end of the patient's airway, e.g., across the glottis of the patient. The conforming cuff or seal 30 is formed by a soft cuff material that bears up against the tissues of the patient and seals against the tissues. The properties of the cuff material enable it to conform and adapt to the shape of the internal anatomical surfaces against which it abuts to seal the mask 30 against the patient. The cuff is not inflatable and therefore does not have a cuff inflation line.

The LMA device 10 also includes an oropharyngeal suction conduit 40 separate from the ventilation conduit 12 (and parallel thereto) that enables a clinician to apply intermittent suction to for removing secretions from the oropharyngeal surface 24 of the mask 20. The oropharyngeal suction conduit 40 has a pipe coupling formation or connector 90 at its upper end as illustrated in the drawings. The connector 90 is a male pipe coupling formation for releasable connection to a complementary female pipe coupling formation 92 on an external suction conduit 70.

The male pipe coupling formation 90 comprises a spigot having transverse ribs that is configured to be received within an industry standard female pipe coupling formation 92, e.g., a 50 mm diameter pipe coupling like those used in the catheter arts.

The LMA device 10 also has an orogastric conduit or orogastric port (gastric drain) 80 for removing gastric matter including reflux from the oesophagus (which is not provided in the LMA device in FIGS. 3 to 8). The orogastric conduit 80 helps to avoid gastric secretions being aspirated or drawn into the lungs, where they can cause complications that are well known and have been described above.

The orogastric conduit 80 has an operatively lower end 81 having an opening that opens to the oesophagus and is positioned outside of the respiratory tree. It faces down below the mask of the LMA device into the oesophagus towards the stomach. The orogastric conduit 80 is separate from both the ventilation conduit 12 and the oropharyngeal suction conduit 40 and does not communicate with either of these conduits 12 and 40 along their length.

The orogastric conduit 80 has an operatively upper end 82 which passes up and out of the patient's mouth and can be operatively coupled to an external suction conduit 70 outside the patient's body. The orogastric conduit 80 can be used to remove gastric secretions, which are sucked up through the orogastric conduit 80 by the application of suction through the external suction conduit 70. The orogastric conduit 80 is formed of a flexible tube like the ventilation conduit 12 and broadly follows the path of the ventilation conduit 12 as it travels from the mask 20 up to its operatively upper end 16.

The orogastric conduit 80 has a connector or pipe coupling formation 91 at its upper end 82 as illustrated in the drawings. The pipe coupling formation 91 is basically the same as the pipe coupling formation 90 on the upper end of the oropharyngeal suction conduit 40. In particular, the pipe coupling formation 91 has a suction control formed by a branch line 94 with an open end that can be closed off by a clinician to apply suction to the orogastric conduit 80.

In the description above, the orogastric conduit 80 with fitted pipe coupling formation 91 can be used as a suction conduit to directly remove gastric matter. The gastric matter is received directly within the conduit 91 itself which is used to channel and direct the gastric matter to the operatively upper end 82. In this application the gastric conduit 80 is not being used as a port to guide insertion of a separate catheter which forms the conduit for withdrawing gastric contents.

However, it must be understood that the orogastric conduit 80 can also be used as a port (or receiving lumen or guide) for providing a passageway for receiving a small catheter therein for removing gastric matter from the patient. The catheter can be fed down through the orogastric port and then down through the operatively lower end of the port. Typically, the catheter is inserted down into the oesophagus and optionally also into the stomach. Such a catheter will necessarily be a small catheter, e.g., an 8 Fr down to a 12 Fr, to enable it to be received with clearance within the orogastric conduit 80.

The collector 62 mounted on the oropharyngeal surface of the mask 20 in FIGS. 9 to 13 has some differences to the collector 62 on the first embodiment described above. The collector 62 has a perimeter region extending around the peripheral edge 31 of the mask 20. The collector 62 also has a raised inner region that is positioned inward of the perimeter region. The raised inner region has a high line extending along a length thereof and slopes down from either side of the high line to the perimeter region on each side of the high line.

The collector 62 has a plurality of suction openings 64 arranged in a line along the perimeter region. In the illustrated embodiment, the collector 62 has at least 10 suction openings 64, e.g., at least 18 suction openings arranged on the perimeter region.

Further, the perimeter region is also configured to form a recessed groove or channel 67 which is shown most clearly in the sectional view shown in FIG. 12 for pooling secretions and directing them to the suction openings 64 formed in the perimeter zone.

In the illustrated embodiment in FIGS. 9 to 13, the channel 67 extends along a substantial portion of the length of the perimeter region.

The collector 62 also includes a further plurality of suction openings 64 in the raised inner region thereof. Applicant believes that additional suction openings 64 on the raised inner region will help to draw substantially all body secretions into the suction conduit 40 and thereby avoid the risk of them being drawn into the airways or trachea. In the illustrated embodiment in FIGS. 9 to 13, these further suction openings 64 comprise suction openings 64 on either side of the high line. Further, these suction openings 64 also extend from an operatively upper end of the mask 20 adjacent to the ventilation conduit 12 to an operatively lower end of the mask 20 remote from the conduit 12. Once again, it needs to be understood that this is merely an example arrangement of suction openings 64 and a variety of other arrangements could also be used. It will be readily understood by persons skilled in the art that the design of the suction openings depends on the size of the LMA which, in turn, depends on what fits the patient. It also depends on the style of LMA, the size of the LMA, the material that is used and achieving a suitable structural integrity for the collector.

Further, Applicant points out that the collector 62 is configured with gently curving surfaces having round edges. The collector 62 does have any square edges let alone any sharp edges which enables the mask 20 with collector 62 to conform to the patient's anatomy. This helps to avoid any trauma to the patient when the LMA device is inserted into and withdrawn from a patient in use.

As shown in the drawings, the suction conduit 40 and the collector 62 extending across the oropharyngeal surface 24 of the mask 20 are integrally formed with the rest of the device 10 and the collector 62, for example, is embedded into the mask 20 of the LMA device 10 during manufacture. Further, as shown in FIGS. 9 and 10, the suction conduit 40 is encased within a housing or sheath 98 that also contains the air tube or ventilation conduit 12 and the orogastric conduit 80. This conveniently bundles these three conduits 12, 40 and 80 together while keeping the internal conduit spaces strictly separate from each other. The housing 98 has a broadly circular or oval shaped cross-sectional configuration.

In use, the LMA device 10 in FIGS. 9 to 12 is used in a similar manner to the embodiment described above with reference to FIGS. 3 to 8. Therefore, the description above with reference to FIGS. 3 to 8 is also applicable here.

The LMA is inserted into a patient in the same way as the LMA device of FIG. 3. Once installed in position, the LMA is ready for use. This is because it has a body conforming cuff that naturally seals against the body tissues of the patient and does not have an inflatable cuff.

In the embodiment illustrated in FIGS. 9 to 12, the suction conduit 40 is releasably coupled to the further external suction conduit 70 by means of complementary male and female pipe coupling formations on each of the suction conduit 40 and the further external suction conduit 70. This enables the attending clinician to apply suction as and when required to the suction conduit 40 and the suction openings 64 on the collector 62. As described above, a suction control is provided by the clinician placing their finger over the open end of the branch line or pipe 94 on the male pipe coupling formation 90 to draw air through the suction conduit 40 instead of from the outside.

Later, if the clinician desires to apply suction to the orogastric conduit 80, then the further suction conduit 70 is manually detached or uncoupled from the suction conduit 40 and connected instead to the orogastric conduit 80. This action of detachment and then reattachment to the orogastric conduit 80 can be quickly and easily manually accomplished by the clinician. This then enables the clinician to apply a suction to the orogastric conduit 80 and withdraw gastric reflux or secretions up through the orogastric conduit 80 and out through the mouth of the patient. Thus, a single external suction conduit 70 which is operatively coupled to a source of suction or vacuum can be used to provide suction to both the suction conduit 40 and the orogastric conduit 80 and this confers a working advantage.

In yet another embodiment of the LMA device that is not illustrated in this specification, the LMA device has an orogastric suction conduit in parallel with a ventilation conduit. It also has a collector extending across the oropharyngeal surface of the mask for drawing secretions into the suction tube. The structure and function of these components is similar to the corresponding components described above on the illustrated embodiments.

The LMA device also has an inflatable cuff around the peripheral edge of the mask like that shown in the embodiment in FIGS. 3 to 8. The cuff is inflated by means of an air line and a valve in the same way as the earlier embodiment.

The LMA device further has an orogastric conduit for extending down to the oesophagus and, optionally, also the stomach for removing gastric contents. The orogastric conduit 80 is very similar to the conduit 80 described above for the embodiment described above with reference to FIGS. 9 to 13.

In addition, this embodiment also includes a bite block. A bite block circumferentially surrounds the ventilation conduit and optionally also the oropharyngeal suction conduit and orogastric conduit and provides a block which the patient to bite on when the LMA device 10 is inserted down their throat and into position. The bite block thus helps to locate the LMA device in position and to maintain it in position.

In use, the LMA device described above but which has not been illustrated, functions in a very similar way to the prior embodiments described above. Further, it is utilized by clinicians in a very similar way to that described above with reference to FIGS. 3 to 8 and FIGS. 9 to 13.

One advantage of the LMA device described above in the detailed description with reference to FIGS. 3 to 8 and 9 to 13 of the drawings, is that it enables secretions in the body cavity adjacent the oropharyngeal surface of the mask to be withdrawn through the oropharyngeal suction conduit. This avoids aspiration of these secretions into the patient's lungs particularly when the LMA device is removed from the patient after a procedure. This helps to avoid respiratory complications, such as bronchospasm and laryngospasm, which can be life threatening. Therefore, the LMA device helps to reduce the risk and occurrence of respiratory complications to patients being treated with an LMA device.

Another advantage of the LMA device described above in the detailed description with reference to FIGS. 3 to 8 and 9 to 13 of the drawings, is that the LMA device has been modified to provide an oropharyngeal suction conduit and collector on the oropharyngeal surface of the mask whereby to implement the invention in a way that does not impose any additional risk or discomfort or hardship on the patient. It is seamlessly integrated into the structure of an LMA device.

Another advantage of the LMA device described above in the detailed description with reference to FIGS. 3 to 8 and 9 to 13 of the drawings, is that the mask and, in particular, the collector on the oropharyngeal surface thereof, have no sharp surfaces. Rather, the surfaces are gently curved and conform to the natural curves and contours of the other features of the mask of the LMA device. Further, the oropharyngeal suction conduit extending from the mask up through the oropharyngeal passage and throat of the patient is relatively unobtrusive and is conveniently bundled up with the ventilation conduit providing the air supply to the trachea of the patient.

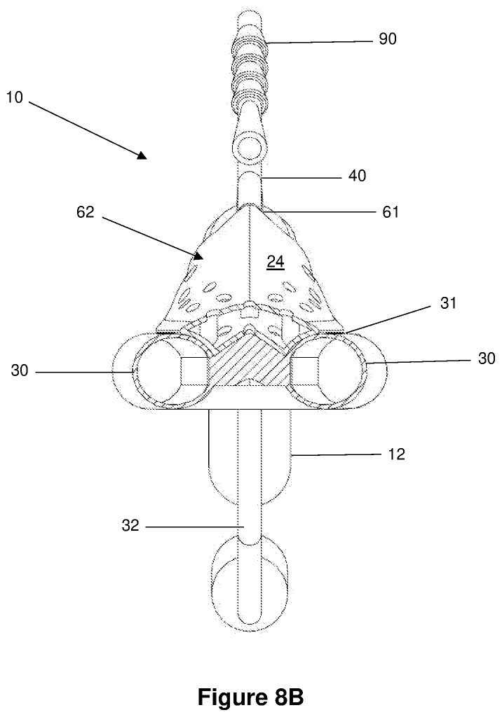

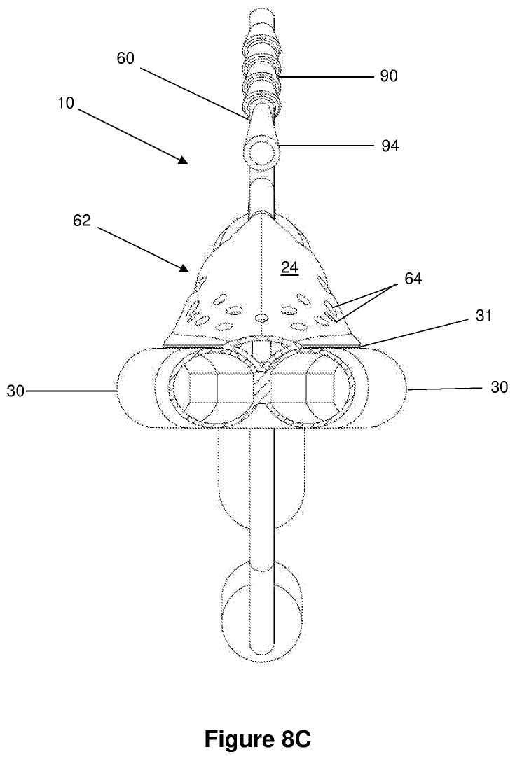

Another advantage of the LMA device described above in the detailed description with reference to FIGS. 8 to 13 of the drawings, is that it provides an orogastric conduit that directly sucks gastric material out of the patient and does not require a separate catheter. The device in this application provides more than a basic orogastric port that requires insertion of a catheter through the port before it can be used. This confers the working advantage of speed of use which in an emergency is advantageous. It also makes it easier for a clinician to access and remove gastric contents when using the LMA device.

Another advantage of the LMA described above in the detailed description with reference to FIGS. 8 to 13 of the drawings, is that the oropharyngeal suction conduit is fitted with a pipe coupling formation that enables it to be quickly and easily coupled to a standard and available external suction conduit in a theatre. Yet further, the orogastric conduit is also fitted with the same pipe coupling formation which enables it to be coupled to the same external suction conduit. The two conduits can be interchangeably coupled to a single external suction conduit to provide the clinician with suction in both conduits as and when required.

Yet another advantage of the LMA described above in the detailed description with reference to FIGS. 3 to 13 of the drawings, is that the collector on the mask for collecting secretions can be formed integrally with the mask when the mask is manufactured.

It will of course be realized that the above has been given only by way of illustrative example of the invention and that all such modifications and variations thereto, as would be apparent to persons skilled in the art, are deemed to fall within the broad scope and ambit of the invention as is herein set forth.

1. A laryngeal mask airway device (LMA), comprising:
a ventilation conduit having an operatively upper end and an operatively lower end;
a mask at the operatively lower end of the ventilation conduit, the mask having an inner surface forming a receptacle that is in fluid communication with the ventilation conduit and an outer oropharyngeal surface opposed to said inner surface and facing away from the receptacle; and wherein the mask comprises an air passage wall having the inner surface that is positioned adjacent to the glottis of a patient in use: an oropharyngeal wall providing said outer oropharyngeal surface extending across the air passage wall spaced away from the air passage wall and defining an oropharyngeal space therebetween, the oropharyngeal wall defining a plurality of suction openings and forming the collector for drawing secretions through the suction openings into the oropharyngeal space; at least one internal support for supporting the oropharyngeal wall in fixed spaced relation relative to the air passage wall; a conforming cuff or seal extending around a perimeter of the air passage wall and the oropharyngeal wall, wherein the air passage wall, the oropharyngeal wall, the at least one internal support, and the conforming cuff or seal are integrally formed;
a separate oropharyngeal suction conduit having an operatively upper end, and an operatively lower end forming the collector defining the plurality of suction openings that extend across the oropharyngeal surface of the mask, wherein in use secretions are drawn through the suction openings into the oropharyngeal suction conduit for removal from the patient through the oropharyngeal suction conduit.

2. A laryngeal mask airway device according to claim 1, wherein the collector extends across substantially all of the oropharyngeal surface of the mask, and the plurality of suction openings are spaced apart from each other across the oropharyngeal surface.

3. A laryngeal mask airway device according to claim 1, wherein the collector has a perimeter region extending along at least part of the collector, and a raised inner region inward of the perimeter region.

4. A laryngeal mask airway device according to claim 3, wherein the plurality of suction openings arranged in a line and spaced apart from each other along the perimeter region, and the collector includes a further plurality of suction openings on the raised inner region.

5. A laryngeal mask airway device according to claim 4, wherein the collector further includes a recess extending along a part of the length of the perimeter region for directing secretions towards the suction openings in the perimeter region.

6. A laryngeal mask airway device according to claim 1, wherein the operatively upper end of the oropharyngeal suction conduit includes a pipe coupling formation for releasable coupling to an external suction conduit, and the pipe coupling formation includes a suction control for enabling a clinician to selectively apply a suction to the oropharyngeal suction conduit.

7. A laryngeal mask airway device according to claim 6, wherein the pipe coupling formation comprises a male pipe coupling formation for releasable connecting to a female pipe coupling formation on the external suction conduit whereby in use to facilitate detachment of the pipe coupling formation from, and reattachment of the pipe coupling formation to, the external suction conduit.

8. A laryngeal mask airway device according to claim 1 further including an orogastric conduit having an operatively upper end and an operatively lower end, for removing gastric matter from the patient, wherein the orogastric conduit does not open into or communicate with the oropharyngeal suction conduit, along its length wherein the orogastric conduit includes an orogastric pipe coupling formation for releasable coupling to an external suction conduit, and the orogastric pipe coupling formation includes a suction control for enabling a clinician to selectively apply a suction to the orogastric conduit; wherein the orogastric pipe coupling formation comprises a male pipe coupling formation for releasable connecting to a female pipe coupling formation on the external suction conduit whereby in use to facilitate detachment of the orogastric pipe coupling formation from, and reattachment of the orogastric pipe coupling formation to, the external suction conduit and wherein the orogastric conduit is configured to also form a port for receiving a catheter therethrough for withdrawing gastric matter from the patient.

9-11. (canceled)

12. A laryngeal mask airway device according to claim 1 wherein the cuff is an inflatable cuff that is inflated for sealing the mask to the body of a patient, the cuff further including an inflation line extending away from the cuff for inflating the cuff.

13. A laryngeal mask airway device according to claim 1 wherein the conforming cuff is formed of a conformable material that conforms to and seals against the body of a patient without being inflated.

14-18. (canceled)

19. A laryngeal mask airway device according to claim 6, wherein the suction control is in the form of a branch pipe on the male pipe coupling formation having an open end that is normally open to the outside air for drawing external air into the oropharyngeal suction conduit, and wherein the open end of the branch pipe is closed to apply a suction to the oropharyngeal suction conduit and draw fluid and secretions into the collector.

20. A laryngeal mask airway device (LMA) in accordance with claim 1 wherein
the operatively upper end comprises a pipe coupling formation for releasable coupling to a complementary pipe coupling formation on an external suction conduit; and
a separate orogastric conduit in parallel with the oropharyngeal suction conduit having an operatively lower end and an operatively upper end, and an orogastric pipe coupling formation on the operatively upper end for releasable coupling to a complementary pipe coupling formation on an external suction conduit,
wherein the pipe coupling formations of the oropharyngeal suction and orogastric conduits are configured to engage with the same complementary pipe coupling formation, whereby to enable the oropharyngeal suction and orogastric conduits to be interchangeably coupled to a single external further suction conduit.

21. A laryngeal mask airway device according to claim 20, wherein the pipe coupling formations of the oropharyngeal suction and orogastric conduits are male pipe coupling formations that are configured to be received within a standard female pipe coupling formation on the external suction conduit.

22-23. (canceled)

24. A mask for a laryngeal mask airway device (LMA), the mask comprising:
an air passage wall that is positioned adjacent to the glottis of a patient in use;
an oropharyngeal wall extending across the air passage wall spaced away from the air passage wall and defining an oropharyngeal space therebetween, the oropharyngeal wall defining a plurality of suction openings and forming a collector for drawing secretions through the suction openings into the oropharyngeal space;
at least one internal support for supporting the oropharyngeal wall in fixed spaced relation relative to the air passage wall.

25. A mask in accordance with claim 24 wherein the air passage wall, the oropharyngeal wall, the at least one internal support, and the conforming cuff or seal are integrally formed.

26. A mask in accordance with claim 24 wherein the plurality of suction openings may be distributed across the surface of the oropharyngeal wall for collecting secretions from across the full surface thereof.

27. A mask in accordance with claim 24 wherein each of the air passage wall and the oropharyngeal wall may have a perimeter and the internal support/s are spaced inward from the perimeter of each of these walls.

28. A mask in accordance with claim 24 wherein at least one internal support is located substantially centrally within the oropharyngeal space.

29. A mask in accordance with claim 24 further comprising a side wall extending between the air passage wall and the oropharyngeal wall around the perimeter of the walls.

30. A mask in accordance with claim 24 further comprising: a conforming cuff or seal extending around a perimeter of the air passage wall and the oropharyngeal wall.
